(19) United States
(12) Patent Application Publication
(10) Pub. No.: US 2023/0103878 A1
(43) Pub. Date: Apr. 6, 2023

(54) HEATING ASSEMBLY AND ATOMIZATION DEVICE

(71) Applicant: Shenzhen Transpring Technology Co., Ltd., Shenzhen, Guangdong (CN)

(72) Inventors: Chuhui CHEN, Shenzhen, Guangdong (CN); Xiuhong ZHANG, Shenzhen, Guangdong (CN)

(73) Assignee: Shenzhen Transpring Technology Co., Ltd., Shenzhen, Guangdong (CN)

(21) Appl. No.: 17/911,054

(22) PCT Filed: Dec. 30, 2021
(86) PCT No.: PCT/CN2021/143098

§ 371 Date: Sep. 12, 2022

(30) Foreign Application Priority Data
Jan. 19, 2021 (CN) 202110072890.7

(51) Int. Cl. A24F 40/46; A24F 40/10; A24F 40/44; A24F 40/485; A24F 40/42; H05B 3/06

(52) U.S. Cl. CPC A24F 40/46; A24F 40/10; A24F 40/44; A24F 40/485; A24F 40/42; H05B 3/06; H05B 2203/016; H05B 2203/021; A61M 11/042

(57) ABSTRACT

The present disclosure provides a heating assembly and an atomization device. The atomization device includes an oil guiding member and a heating member. The oil guiding member has a through hole cutting through the oil guiding member. The through hole is configured to guide atomized gas generated on the oil guiding member out of the oil guiding member. The oil guiding member includes a connecting part. The connecting part is located in the through hole. The connecting part spans the through hole along a radial direction of the through hole. The heating member is embedded in one end of the oil guiding member. At least part of the heating member is located on the connecting part. The heating member is used for heating the oil guiding member, so that the liquid to be atomized is atomized on an end face of the oil guiding member close to the heating member.

### CROSS-REFERENCE TO RELATED APPLICATION(S)

This application is a U.S. National Stage filing under 35 U.S.C. § 371 of International Patent Application No. PCT/CN2021/143098, filed on Dec. 30, 2021, which in turn claims priority under PCT Article 8 and/or 35 U.S.C. § 119(a) to Chinese Patent Application No. 202110072890.7, filed in the State Intellectual Property Office of China on Jan. 19, 2021, and which the title is “porous ceramic heating assembly and atomization assembly”, the entire contents of which are incorporated by reference herein.

### TECHNICAL FIELD

The present disclosure relates to technical fields of atomization, in particular to a heating assembly and an atomization device.

### BACKGROUND

The application of atomization devices in modern life is more and more extensive, such as medical atomizers, air humidifiers, micro electronic atomizers, and the like. With the continuous expansion of the market, the market has higher and higher requirements for the comprehensive performance of the atomization devices, especially reflected in the further improvement of the requirements for performance such as heating efficiency, atomization quality, and atomization reduction degree.

Some existing atomization devices have problems such as uneven heating, insufficient atomization and the like, which in turn lead to poor atomization quality and atomization effect when the atomization device atomizes the liquid to be atomized. At the same time, the gas generated by atomization is not separated from the liquid to be atomized, which leads to the mixing of the gas generated by atomization with the liquid to be atomized during a release process, which reduces the reduction degree of atomization and affects the quality of atomization. In the medical industry, in order to achieve full atomization of the drugs to be atomized, the requirements for atomization quality and atomization reduction degree are higher. For micro electronic atomizer, the efficiency of the heating assembly is very important. Unstable heating efficiency or low heating efficiency will directly affect the continuity of the gas generated by the atomization and seriously affects the experience of a user.

Therefore, how to design a heating assembly and an atomization device that can realize gas-liquid separation, high atomization reduction degree, high atomization efficiency and good atomization quality has become an urgent technical problem to be solved.

### SUMMARY

In order to solve the deficiencies of the prior art, the present disclosure provides a heating assembly and an atomization device which have a high atomization efficiency, a large atomization amount and are capable of realizing gas-liquid separation.

On one hand, the present disclosure provides a heating assembly, which is applied in an atomization device and used for heating and atomizing liquid to be atomized, including an oil guiding member and a heating member.

The oil guiding member defines a through hole cutting through the oil guiding member. The through hole is configured to guide atomized gas generated on the oil guiding member out of the oil guiding member. The oil guiding member includes a connecting part. The connecting part is located in the through hole. The connecting part spans the through hole along a radial direction of the through hole.

The heating member is embedded in one end of the oil guiding member. At least part of the heating member is located on the connecting part. The heating member is used for heating the oil guiding member, so that the liquid to be atomized is atomized on an end face of the oil guiding member close to the heating member.

Optionally, the oil guiding member includes a main body part and an extension part that are coupled to each other. The through hole cuts through the extension part and the main body part in sequence. The main body part includes a bearing surface and an atomizing surface arranged opposite to each other. The bearing surface includes a connecting surface and a liquid absorbing surface that are connected. The extension part is fixedly arranged on the connecting surface. The connecting part is arranged at an opening of the through hole close to the atomizing surface. The liquid to be atomized flows to the atomizing surface through the liquid absorbing surface, and is atomized on the atomizing surface.

Optionally, the heating member includes a heating body and a positive electrode contact and a negative electrode contact electrically coupled to both ends of the heating body. The heating body is arranged on the atomizing surface in an arc shape or a curve shape. The arc corresponding to the heating body is greater than or equal to 180°, and at least part of the heating body is located on the connecting part.

Optionally, the heating member includes a heating body and a positive electrode contact and a negative electrode contact electrically coupled to both ends of the heating body. The positive electrode contact and the negative electrode contact are arranged on opposite sides of the through hole along a radial direction of the through hole. The positive electrode contact and the negative electrode contact are located close to a first end and a second end of the connecting part respectively. A first part of the heating body extends from the positive electrode contact along the atomizing surface to the second end of the connecting part. A second part of the heating body extends from the second end of the connecting part to the first end of the connecting part. A third part of the heating body extends from the first end of the connecting part along the atomizing surface to the second end of the connecting part and then connects the negative electrode contact.

Optionally, the extension part includes a sleeve and at least one protrusion located on an outer peripheral surface of the sleeve. The at least one protrusion abuts against the atomizing surface. The sleeve, the at least one protrusion and the main body are integrally formed. The main body further defines a plurality of receiving grooves. Openings of the receiving grooves are defined on the atomizing surface. At least part of the bottom of the receiving groove extends to the protrusion. The plurality of the the receiving grooves are configured for receiving the positive electrode contact and the negative electrode contact respectively.

On the other hand, the present disclosure also provides an atomization device. The atomization device includes the heating assembly.

Optionally, the atomization device further includes an atomizing rod and a sealing seat. The atomizing rod includes a receiving seat. The heating assembly, the sealing seat and the receiving seat are sequentially sleeved from inside to outside. A part of the sealing seat is sealed between one side of the extension part away from the main body and the receiving seat, and another part of the sealing seat is sealed between a peripheral side wall of the extension part and the receiving seat, and another part of the sealing seat is sealed between a peripheral side wall of the main body and the receiving seat. An peripheral side wall of the extension part, the liquid absorbing surface, and an inner side wall of the sealing seat cooperatively form a receiving space. The receiving seat defines at least one liquid guiding hole. The sealing seat defines at least one liquid inlet hole. The liquid guiding hole, the liquid inlet hole and the receiving space are interconnected in sequence. The liquid to be atomized passes through the at least one liquid guiding hole, the at least one liquid inlet hole and the receiving space in sequence and then flows to the liquid absorbing surface.

Optionally, the atomizing rod further includes a main rod body, which is integrally formed with the receiving seat. An inner cavity of the main rod body, a part of an inner cavity of the receiving seat, and a part of an inner cavity of the sealing seat and the through hole are sequentially interconnected. The atomized gas generated on the atomizing surface passes through the through hole, the part of the inner cavity of the sealing seat, the part of the inner cavity of the receiving seat, and the inner cavity of the main rod body, and then flows out.

Optionally, the atomization device further includes a suction nozzle and an oil cup. The suction nozzle is sleeved and sealed on a periphery of an end of the main rod body away from the receiving seat. The oil cup is sleeved on the periphery of the main rod body. One end of the oil cup is coupled to the suction nozzle hermetically, and the other end of the oil cup is coupled to an periphery of the receiving seat away from the main rod body hermetically. An inner side wall of the oil cup and an outer side wall of the atomizing rod cooperatively form a liquid storage cavity for storing the liquid to be atomized. The at least one liquid guiding hole is interconnected with the liquid storage cavity.

Optionally, the atomization device further includes an atomization base, an atomization outer ring and an oil absorbing member. One end of the atomization base is received in the inner cavity of the receiving seat and abuts against the sealing seat, and the other end extends out of the receiving seat. An inner cavity of the atomization base is coupled to the through hole. The oil absorbing member is arranged in the atomization base, and is used to absorb un-atomized liquid that is to be atomized on the atomizing surface. The atomizing outer ring is fixedly arranged on an periphery of the end of the oil cup away from the suction nozzle, an periphery of the end of the receiving seat away from the main rod body, and an periphery of the atomization base.

Optionally, the atomization device further includes a battery assembly. The battery assembly is electrically coupled to the heating member. The battery assembly is coupled to an end of the atomization base extending out of the atomization outer ring.

The heating assembly and the atomization device provided by the present disclosure are provided with the through hole on the oil guiding member, so that the atomized gas generated after the atomizing surface heats and atomizes the liquid to be atomized is released through the through hole cutting through the oil guiding member, to avoid the atomized gas mixing with the liquid to be atomized during release, the atomization reduction degree of the heating assembly and atomization device is higher. The oil guiding member includes a connecting part arranged in the through hole. The heating member is arranged at one end of the oil guiding member, and at least part of the heating member is arranged on the connecting part, such that the position corresponding to the through hole can also be heated by the heating member, to atomize the liquid to be atomized, thereby making the heating assembly have a higher atomization efficiency, a better atomization effect, a larger atomization amount, and a more uniform atomization .

### BRIEF DESCRIPTION OF THE ACCOMPANYING DRAWINGS

In order to illustrate the technical solutions of the embodiments of the present disclosure more clearly, the accompanying drawings required in the embodiments will be briefly introduced below.

FIG. 1 illustrates a schematic structural diagram of an atomization device, according to one embodiment of the present disclosure;

FIG. 2 illustrates an exploded view of the atomization device, according to one embodiment of the present disclosure;

FIG. 3 illustrates a cross-sectional view of the atomization device, according to one embodiment of the present disclosure;

FIG. 4 illustrates a schematic structural diagram of an atomizing rod, according to one embodiment of the present disclosure;

FIG. 5 illustrates a schematic structural diagram of a sealing seat, according to one embodiment of the present disclosure;

FIG. 6 illustrates a cross-sectional view of the sealing seat, according to one embodiment of the present disclosure;

FIG. 7 illustrates a schematic structural diagram of a heating assembly, according to one embodiment of the present disclosure;

FIG. 8 illustrates a top view of the heating assembly, according to one embodiment of the present disclosure;

FIG. 9 illustrates a schematic diagram of a bearing surface, according to one embodiment of the present disclosure;

FIG. 10 illustrates a schematic diagram of an atomizing surface, according to one embodiment of the present disclosure;

FIG. 11 illustrates a bottom view of a first heating member, according to the embodiment of the present disclosure;

FIG. 12 illustrates a cross-sectional view of a heating assembly, according to one embodiment of the present disclosure;

FIG. 13 illustrates a bottom view of a second heating member, according to the embodiment of the present disclosure;

FIG. 14 illustrates a bottom view of a third heating member, according to the embodiment of the present disclosure;

FIG. 15 illustrates a bottom view of a fourth heating member, according to the embodiment of the present disclosure;

FIG. 16 illustrates a bottom view of a fifth heating member, according to the embodiment of the present disclosure.

### DETAILED DESCRIPTION OF ILLUSTRATED EMBODIMENTS

The technical solutions in the embodiments of the present disclosure will be clearly and completely described below with reference to the accompanying drawings in the embodiments of the present disclosure. The embodiments of the present disclosure are adaptively combined with each other, and new embodiments formed by the combination are also within a protection scope of the present disclosure.

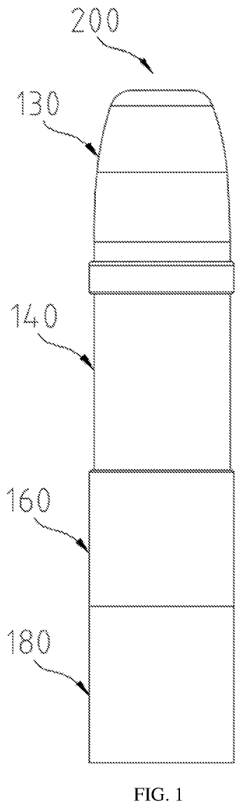

Please referring to FIG. 1, FIG. 1 illustrates a schematic structural diagram of an atomization device, according to one embodiment of the present disclosure.

The technical solutions of the present disclosure are described in detail in the embodiments of the present disclosure, by taking a cylindrical micro electronic atomizer as an example. The cylindrical micro electronic atomizer is roughly in a shape of a long cylinder. The cylindrical micro electronic atomizer has characteristics of easy portability, strong applicability and wide application range, and the like. However, the atomization device 200 of the present disclosure is applicable to, but not limited to, an atomization device such as an air humidifier, a medical nebulizer, a micro nebulizer, and the like.

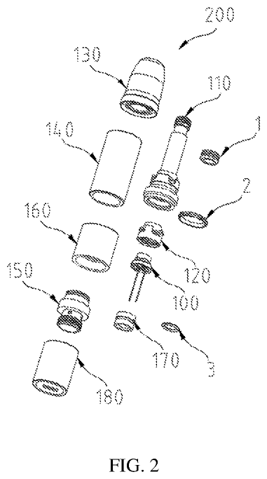

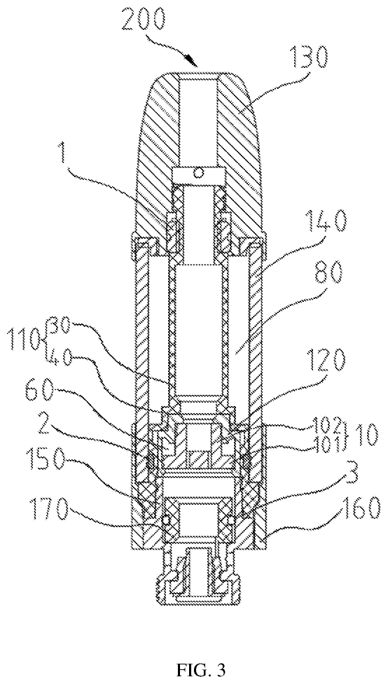

Please referring to FIGS. 2 and 3, FIG. 2 illustrates an exploded view of an atomizing assembly, according to one embodiment of the present disclosure; and FIG. 3 illustrates a cross-sectional view of an atomizing assembly, according to one embodiment of the present disclosure.

The atomization device 200 includes a heating assembly 100, an atomizing rod 110, a sealing seat 120, a suction nozzle 130, an oil cup 140, an atomization base 150, an atomizing outer ring 160, an oil absorbing member 170 and a battery assembly 180.

The heating assembly 100 is located in one end of the atomizing rod 110.

The sealing seat 120 is located in the atomizing rod 110, and the sealing seat 120 is sealed between the heating assembly 100 and the atomizing rod 110.

The suction nozzle 130 is coupled to the other end of the atomizing rod 110 hermetically (the end of the atomizing rod 110 away from the heating assembly 100).

The oil cup 140 is sleeved on a periphery of the atomizing rod 110, and an inner side wall of the oil cup 140 is spaced from a part of the outer side wall of the atomizing rod 110. One end of the oil cup 140 close to the suction nozzle 130 is hermetically coupled to the suction nozzle 130 through a first sealing ring 1. An inner wall of the end of the oil cup 140 close to the heating assembly 100 is hermetically coupled to an outer wall of the atomizing rod 110 through a second sealing ring 2.

One end of the atomization base 150 close to the heating assembly 100 is located in the atomizing rod 110, and the end of the atomization base 150 close to the heating assembly 100 abuts against the sealing seat 120.

The atomizing outer ring 160 is sequentially sleeved on an outer side wall of one end of the oil cup 140 close to the heating assembly 100, an outer side wall of one end of the atomizing rod 110 away from the suction nozzle 130, and an outer side wall of one end of the atomization base 150 away from the sealing seat 120, such that the oil cup 140, the atomizing rod 110 and the atomization base 150 are fixedly coupled.

The oil absorbing member 170 is located in the atomization base 150. The oil absorbing member 170 is located opposite to the heating assembly 100, for absorbing un-atomized liquid that is to be atomized by the heating assembly 100, which can prevent the liquid to be atomized from entering other components of the atomization device 200 and affecting the normal work of the atomization device 200. The material of the oil absorbing member 170 includes, but is not limited to, oil absorbing materials such as oil absorbing ceramics, oil absorbing sponge and the like. The outer side wall of the oil absorbing member 170 and the inner side wall of the atomization base 150 are hermetically coupled through a third sealing ring 3, to reduce an occurrence of leakage. The leakage refers to that the liquid, to be atomized, which is atomized incompletely, is leaked from a gap between an outer peripheral side wall of the oil absorbing member 170 and an inner side wall of the atomization base 150.

The battery assembly 180 is coupled to one end of the atomization base 150 extending from the atomizing outer ring 160 and away from the suction nozzle 130. The connecting way of the battery assembly 180 and the atomization base 150 can include, but is not limited to, a screw connection, a snap connection and other connection methods. The battery assembly 180 is electrically coupled to the heating assembly 100, so that the heating assembly 100 heats and atomizes the liquid to be atomized, thereby generating the atomized gas.

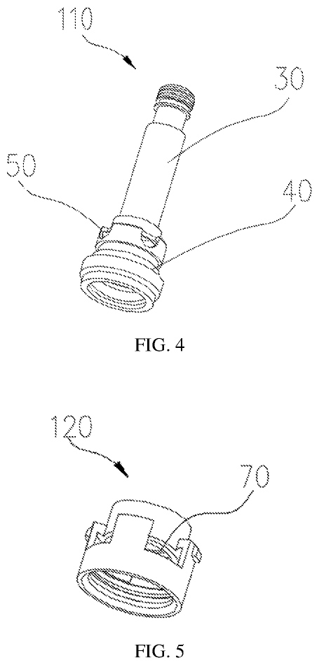

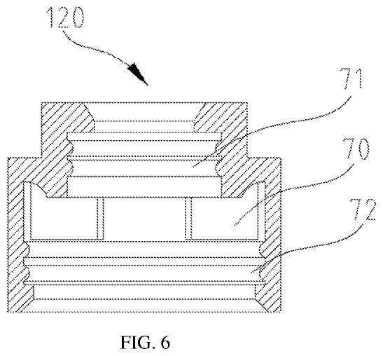

Please referring to FIG. 4, FIG. 5 and FIG. 6, FIG. 4 illustrates a schematic structural diagram of an atomizing rod, according to one embodiment of the present disclosure, FIG. 5 illustrates a structural schematic diagram of a sealing seat, according to one embodiment of the present disclosure, and FIG. 6 illustrates a cross-sectional view of a sealing seat, according to one embodiment of the present disclosure.

The atomizing rod 110 has a hollow structure and includes a main rod body 30 and a receiving seat 40. The main rod body 30 and the receiving seat 40 are made of same materials and interconnected with each other. One end of the main rod body 30 is interconnected with the suction nozzle 130. The receiving seat 40 is located at one end of the atomizing rod 110 away from the suction nozzle 130. The sealing seat 120 is received in the receiving seat 40 and is hermetically coupled to the inner side wall of the receiving seat 40. The heating assembly 100 is received in the sealing seat 120, and a part of the heating assembly 100 is hermetically coupled to the inner wall of the sealing seat 120. A structure is formed in which the heating assembly 100, the sealing seat 120 and the receiving seat 40 are sequentially sleeved from the inside to the outside.

One end of the oil cup 140 close to the suction nozzle 130 is hermetically coupled to the suction nozzle 130 through the first sealing ring 1. The other end of the oil cup 140 away from the suction nozzle 130 is hermetically coupled to the outer side wall of the receiving seat 40 through the second sealing ring 2. The main rod body 30 is received in the oil cup 140. The outer side wall of the main rod body 30 is spaced apart from the inner side wall of the oil cup 140 to form a liquid storage chamber 80. The end of the oil cup 140 close to the suction nozzle 130 is hermetically coupled to the suction nozzle 130 through the first sealing ring 1; the other end of the oil cup 140 away from the suction nozzle 130 is hermetically coupled to the outer side wall of the receiving seat 40 through the second sealing ring 2, so that the liquid sealing and storage performance of the liquid storage chamber 80 is better.

One end of the atomization base 150 is received at the end of the receiving seat 40 away from the suction nozzle 130, and the end of the atomization base 150 received in the receiving seat 40 abuts the end of the sealing seat 120 away from the suction nozzle 130.

The inner cavity of the suction nozzle 130, the inner cavity of the main rod body 30, the inner cavity of the receiving seat 40, and at least part of the inner cavity of the sealing seat 120 are interconnected in sequence along the axial direction, so that the gas generated after the heating assembly 100 atomizes the liquid, to be atomized, flows, along at least part of the inner cavity of the sealing seat 120, at least part of the inner cavity of the receiving seat 40, the inner cavity of the main rod body 30 and the inner cavity of the suction nozzle 130 sequentially and then is released to outside of the atomization device 200, to form an atomized gas channel.

The atomizing rod 110 further defines at least one liquid guiding hole 50. The at least one liquid guiding hole 50 is defined on the receiving seat 40. The at least one liquid guiding hole 50 cuts through the receiving seat 40 along the radial direction of the receiving seat 40. One side of the liquid guiding hole 50 is coupled to the liquid storage chamber 80, so that the liquid to be atomized in the liquid storage chamber 80 flows into the receiving seat 40, and is then atomized at the heating assembly 100 accommodated in the receiving seat 40 to form atomized gas. The at least one liquid guiding hole 50 is four through holes evenly distributed in a middle section of the receiving seat 40. The liquid inlet hole 70 on the sealing seat 120 is defined corresponding to the position of the liquid guiding hole 50 on the receiving seat 40.

The inner side wall of the sealing seat 120 has a first sealing part 71 and a second sealing part 72. One end of the heating assembly 100 is hermetically coupled to the inner side wall of one end of the sealing seat 120 through the first sealing part 71, and the other end of the heating assembly 100 is hermetically coupled to the inner side wall of the middle part of the sealing seat 120 through the second sealing part 72. An receiving space 60 is formed between the sealing seat 120 and the heating assembly 100. By arranging the first sealing part 71 and the second sealing part 72, the gas generated by atomization will not be lost along the inner wall of the sealing seat 120. The atomization gas, generated by the atomization of the liquid to be atomized, is guaranteed to be completely released.

The sealing seat 120 also defines at least one liquid inlet hole 70 that interconnects with the receiving space 60. The liquid to be atomized in the liquid storage chamber 80 enters the receiving seat 40 through the liquid guiding hole 50, and the liquid to be atomized entering the receiving seat 40 enters the receiving space 60 through the liquid inlet hole 70. The liquid storage chamber 80, the liquid guiding hole 50, the liquid inlet hole 70 and the receiving space 60 are interconnected in sequence to form a channel for the liquid to be atomized. The liquid to be atomized entering the receiving space 60 flows to the heating assembly 100 for heating and atomizing to form the atomized gas.

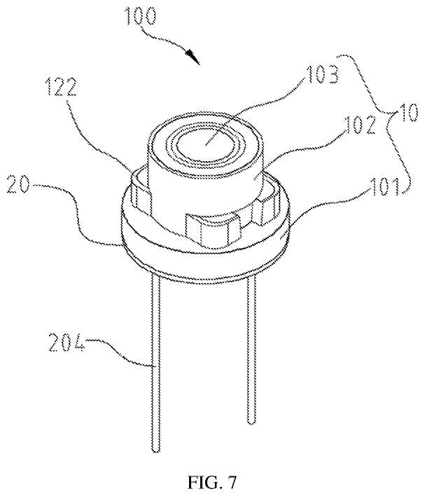

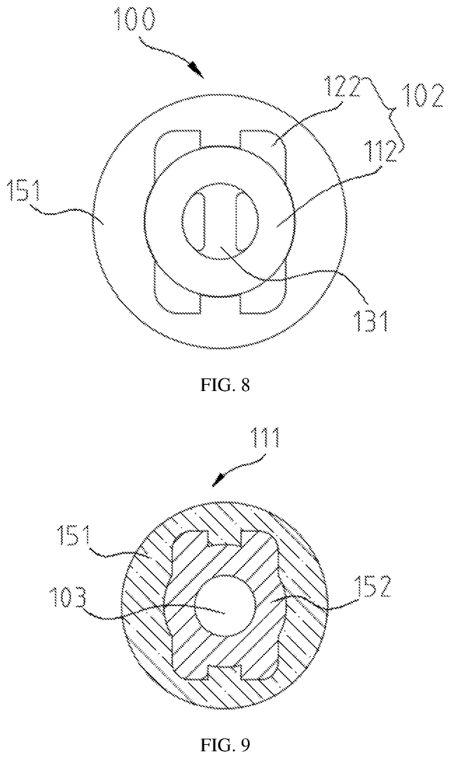

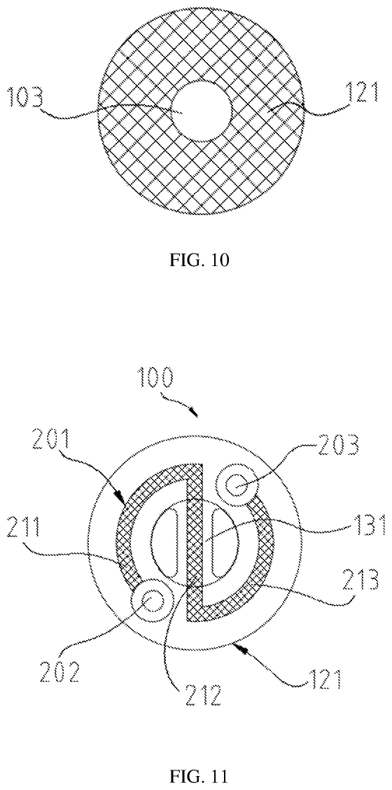

Please referring to FIG. 7, FIG. 8, FIG. 9 and FIG. 10, FIG. 7 illustrates a schematic structural diagram of a heating assembly, according to one embodiment of the present disclosure; FIG. 8 illustrates a top view of a heating assembly, according to one embodiment of the present disclosure; FIG. 9 illustrates a schematic diagram of a bearing surface, according to one embodiment of the present disclosure; and FIG. 10 illustrates a schematic diagram of an atomizing surface, according to one embodiment of the present disclosure.

The heating assembly 100 includes an oil guiding member 10 and a heating member 20. The heating member 20 is fixed to one end of the oil guiding member 10. The materials of the oil guiding member 10 include, but is not limited to, oil guiding materials such as porous ceramics. The oil guiding member 10 defines a plurality of fine holes (not shown in the figure). The plurality of fine holes of the oil guiding member 10 are used to strengthen the oil conduction performance of the oil guiding member 10. The oil guiding member 10 includes a main body 101 and an extension part 102 which are made of same materials. The extension part 102 is located on an end surface of one end of the main body part 101. The main body part 101 includes a bearing surface 111 and an atomizing surface 121 that are disposed backward. The bearing surface 111 is an end surface of an end of the main body part 101 having the extension part 102. The bearing surface 111 includes a liquid absorbing surface 151 and a connecting surface that are coupled together. In detail, the extension part 102 is coupled to the connecting surface 152, and the part of the bearing surface 111 that is not coupled to the extending part 102 is the liquid absorbing surface 151. The liquid absorbing surface 151 is used for bearing the liquid to be atomized. The liquid to be atomized enters the atomizing surface 121 of the oil guiding member 10 through the liquid absorbing surface 151.

Understandably, in one embodiment, an area of the liquid absorbing surface 151 is smaller than an area of the atomizing surface 121, and the area of the liquid absorbing surface 151 is the difference between the area of the bearing surface 111 and the area of the cross section of the connecting surface 152. The area of the liquid absorbing surface 151 is smaller than the area of the atomizing surface 121, so that the liquid absorbing speed of the liquid absorbing surface 151 and the atomizing speed of the atomizing surface 121 can cooperate with each other, so as to realize the full atomization of the liquid to be atomized and improve the atomization quality of the atomizing surface 121. In at least one embodiment, the area of the liquid absorbing surface 151 may be equal to or greater than that of the atomizing surface 121.

The oil guiding member 10 also has a through hole 103. One end of the through hole 103 is defines on the end face of the end of the extension part 102 away from the bearing surface 111, and the other end of the through hole 103 cuts through the atomizing surface 121, such that the atomized gas, generated by the atomizing surface 121, is released into the suction nozzle 130 through the through hole 103, at least part of the inner cavity of the sealing seat 120, at least part of the inner cavity of the receiving seat 40 and the main rod body 30. At the same time, the gas, generated by the atomizing surface 121, is released into the suction nozzle 130 through the through hole 103, and will not mix with the liquid to be atomized in the oil guiding member 10, so as to realize the gas-liquid separation and avoid the reduction of the reduction degree of the gas generated by the atomization. At the same time, the through hole 103 cuts through the extension part 102 and the main body 101, it can effectively prevent the liquid to be atomized from blocking the flow channel of the atomized gas due to insufficient atomization, thereby improving the performance of the heating assembly 100 to heat and atomize the liquid to be atomized to form atomized gas.

The extension part 102 includes an integrally formed sleeve 112 and a plurality of protrusions 122 located on the outer peripheral surface of the sleeve 112. In this embodiment of the present disclosure, the plurality of protrusions 122 are four. The sleeve 112 and the plurality of protrusions 122 are connected with the main body 101 as a whole. The through hole 103 cuts through the sleeve 112 and the main body part 101.

The extension part 102 is formed by the protrusions 122 and the sleeve 112, which causes part of the bearing surface 111 to be formed the liquid absorbing surface 151, and the area of the liquid absorbing surface 151 is smaller than that of the atomizing surface 121, such that the speed of the liquid to be atomized flowing into the liquid absorbing surface 151 matches the speed of the atomizing surface 121 atomizing the liquid to be atomized, the liquid to be atomized can be thus fully atomized, the atomization quality is thus higher, and the reduction degree of the atomized gas generated by the atomization of the liquid to be atomized is thus higher.

The main body part 101 further defines a plurality of receiving grooves. In this embodiment of the present disclosure, the plurality of receiving grooves is two. The plurality of receiving grooves include a first receiving groove 141 and a second receiving groove 142. The openings of the receiving grooves 142 are all set on the atomizing surface 121. At least part of the first receiving groove 141 and at least part of the second receiving groove 142 extend to the inside of the two protrusions 122 respectively. The first receiving groove 141 and the second receiving groove 142 are respectively used to receive the two heating electrodes 204 electrically coupled to the heating member 20.

Specifically, the outer peripheral surface of the extension part 102, the liquid absorbing surface 151 of the main body 101, and the inner wall of the sealing seat 120 cooperatively form the receiving space 60. The liquid guiding hole 50 of the receiving seat 40 is interconnected with the liquid storage cavity 80 and the liquid inlet hole 70 of the sealing seat 120. The liquid inlet hole 70 of the sealing seat 120 is interconnected with the liquid guiding hole 50 of the receiving seat 40 and the receiving space 60, so that the liquid to be atomized flows from the liquid storage chamber 80 to the liquid absorbing surface 151 of the main body 101.

The heating member 20 is fixedly coupled to the end of the main body part 101 away from the extension part 102. Specifically, the heating member 20 is located at the end of the main body part 101 close to the atomizing surface 121. The connecting way between the heating member 20 and the main body part 101 includes, but is not limited to, bonding, snap connection, inlay and other connection methods. The heating member 20 generates heat under the action of the battery assembly 180, so that the atomizing surface 121 of the oil guiding member 10 generates heat to atomize the liquid to be atomized. Through the cooperation of the heating member 20 and the oil guiding member 10 to generate heat and atomize, such that the heating assembly 100 has a higher heating efficiency, a larger atomization amount, and a better atomization effect.

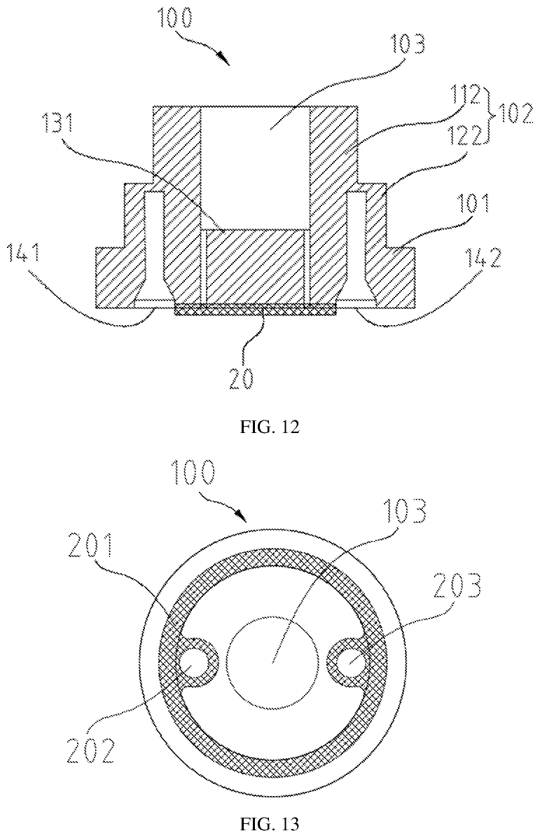

Please referring to FIG. 11 and FIG. 12, FIG. 11 illustrates a bottom view of a first heating member, according to one embodiment of the present disclosure, and FIG. 12 illustrates a cross-sectional view of a heating assembly, according to one embodiment of the present disclosure.

The heating assembly 100 also includes a connecting part 131. The connecting part 131 is arranged in the opening of the through hole 103 close to the atomizing surface 121. One part of the heating member 20 is arranged on the atomizing surface 121, and the other part of the heating member 20 is arranged on the connecting part 131. By arranging the connecting part 131 in the opening of the through hole 103 close to the atomizing surface 121, and disposing the heating member 20 on one side of the connecting part 131 away from the extension part 102, the middle part of the atomizing surface 121 of the main body 101 will also generate heat to atomize the liquid to be atomized, so that the heating of the atomizing surface 121 is more uniform, the heating efficiency is higher, the atomization amount is larger, and the atomization effect is better.

Referring to FIG. 11, in the first heating assembly 100 provided by the embodiment of the present disclosure, the connecting part 131 spans the through hole 103 along the radial direction of the through hole 103, and opposite ends of the connecting part 131 and the inner wall of the through hole 103 are made of same materials. The heating member 20 includes a heating body 201 and a positive electrode contact 202 and a negative electrode contact 203 that are electrically coupled to opposite ends of the heating body 201. The positive electrode contact 202 and the negative electrode contact 203 are respectively located close to the first end and the second end of the connecting part 131.

The first part 211 of the heating body 201 extends from the positive electrode contact 202 along the atomizing surface 121 to the second end of the connecting part 131, and the first part 211 is arc-shaped. The first part 211 of the heating body 201 is embedded on the atomizing surface 121. The second part 212 of the heating body 201 extends from the second end of the connecting part 131 to the first end of the connecting part 131, and the second part 212 is linear. The second part 212 of the heating body 201 is embedded on the side of the connecting part 131 close to the atomizing surface 121. The third part 213 of the heating body 201 extends from the first end of the connecting part 131 along the atomizing surface 121 to the second end of the connecting part 131 and is coupled to the negative electrode contact 203, and the third part 213 is arc-shaped. The third part 213 of the heating body 201 is embedded on the atomizing surface 121.

By embedding the heating body 201 on the atomizing surface 121 and the connecting part 131, the heating body 201 forms an S-shaped bridge design, so that the heating efficiency of a middle section of the heating body 201 (ie, the position close to the through hole 103) is higher, such that the atomization amount of the atomizing surface 121 is larger, and the atomization effect is better.

Optionally, the shape of the connecting part 131 spanning the through hole 103 in the radial direction of the through hole 103 includes, but is not limited to, a linear shape, a curved shape, and the like. The number of connection parts 131 may include, but is not limited to, 1, 2, 3, and the like.

In at least one embodiment, when the number of the connecting parts 131 is multiple, the multiple connecting parts 131 may be arranged in a cross or at intervals, so that more heating bodies 201 can be arranged at the positions where the through hole 103 is located, thereby improving the atomization efficiency and atomization uniformity of the atomizing surface 121.

Optionally, the shapes of the first part 211, the second part 212 and the third part 213 include, but are not limited to, shapes such as straight lines, circular arcs, waves, and combinations thereof.

Optionally, the type of the heating body 201 includes, but is not limited to, heating elements such as a metal coating, a heating sheet, a stainless steel sheet, and the like.

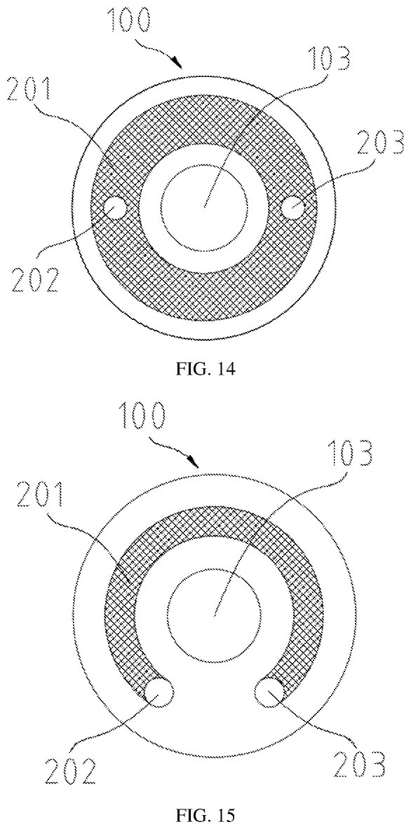

Please referring to FIG. 13 and FIG. 14, FIG. 13 illustrates a bottom view of a second heating assembly, according to one embodiment of the present disclosure, and FIG. 14 illustrates a bottom view of a third heating assembly, according to one embodiment of the present disclosure.

The structure of the second heating assembly 100 provided in the present disclosure is substantially the same as that of the first heating assembly 100 provided in the present disclosure, and the difference is that in the second heating assembly 100 provided in the present disclosure, the connecting part 131 is omitted, and the heating body 201 is in the shape of an end-to-end ring in comparison to the first heating assembly 100 provided in the present disclosure

Specifically, the heating member 20 includes a heating body 201 and a positive electrode contact 202 and a negative electrode contact 203 that are electrically coupled to the two opposite side of the heating body 201. The heating body 201 is in an annular shape, and is arranged on the atomizing surface 121 on the periphery of the through hole 103. The positive electrode contact 202 and the negative electrode contact 203 are provided in an formed space surrounded by the heating body 201. The heating main body 201 is distributed on the atomizing surface 121 in an annular shape, so that the heating of the atomizing surface 121 is more uniform, the atomization effect of the liquid to be atomized is better, thereby reducing a situation of uneven and insufficient atomization of some liquid to be atomized, which further improves the atomization quality and the reduction degree of the atomized gas.

Optionally, the shape of the heating body 201 includes, but is not limited to, a circular ring, an elliptical ring, a polygon connected end to end, a wave shape connected end to end, and the like.

The structure of the third heating assembly 100 provided in the present disclosure is substantially the same as that of the second heating assembly 100 provided in the present disclosure, and the difference is that in the third heating assembly 100 provided in the present disclosure, the positive electrode contact 202 and the negative electrode contact 203 are located within the range covered by the heating body 201 in comparison to the second heating assembly 100 provided in the present disclosure.

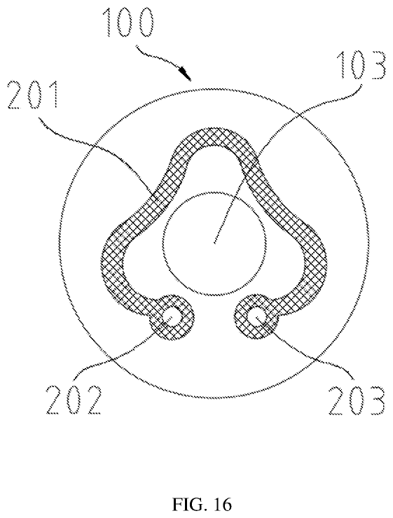

Please referring to FIGS. 15 and 16, FIG. 15 illustrates a bottom view of a fourth heating assembly, according to one embodiment of the present disclosure, and FIG. 16 illustrates a bottom view of a fifth heating assembly, according to one embodiment of the present disclosure.

The structure of the fourth heating assembly 100 provided in the present disclosure is substantially the same as that of the first heating assembly 100 provided in the present disclosure, and the difference is that in the fourth heating assembly 100 provided in the present disclosure the connection part 131 is omitted in comparison to the first heating assembly 100 provided in the present disclosure.

Specifically, the heating member 20 includes a heating body 201 and a positive electrode contact 202 and a negative electrode contact 203 that are electrically coupled to the heating body 201. The positive electrode contact 202 and the negative electrode contact 203 are both arranged on one side of the through hole 103. The heating body 201 is arranged on the atomizing surface 121 on the peripheral of other side of the through hole 103. The corresponding arc of the heating body 201 is greater than 180°. The heating body 201 is arranged on the atomizing surface 121 around the through hole 103 in in a circular arc shape. By directly arranging the arc-shaped heating body 201 on the atomizing surface 121, the structure of the heating member 20 is simpler, and the through hole 103 is not blocked by other components, so that the air guiding function of the through hole 103 is better, thereby reducing the atomized gas mixing with the liquid to be atomized, and the atomization reduction degree is higher.

The structure of the fifth heating assembly 100 provided in the present disclosure is substantially the same as that of the fourth heating assembly 100 provided in the present disclosure, and the difference is that in the fifth heating assembly 100 provided in the present disclosure, the heating body 201 is arranged on the atomizing surface 121 on the other side of the periphery of the through hole 103 in a curved wave shape in comparison to the fourth heating assembly 100 provided in the present disclosure.

In other embodiments, the fourth heating assembly 100 provided by the present disclosure or the fifth heating assembly 100 provided by the present disclosure can be improved, because most of the users hold the product by hands during the use of the product, which may easily lead to uneven distribution of the liquid to be atomized on the atomizing surface 121 due to the inclination of the heating assembly 100, thereby easily affecting the effect of the heating member 20 for heating and atomizing the liquid to be atomized. Therefore, the connecting part 131 is provided in the fourth heating assembly 100 provided by the present disclosure or the fifth heating assembly 100 provided by the present disclosure, and at least part of the heating body 201 is embedded on the connecting part 131, so that the heating body 201 has an overall shape of “M” shape, the user can rotate the product so that the side with more liquid to be atomized on the atomizing surface 121 corresponds to the part of the heating main body 201 located on the connecting part 131, so as to ensure that the liquid to be atomized can be fully atomized to form atomized gas, thereby improving the atomization quality.

The above are some embodiments of the present disclosure. It should be pointed out that for those of ordinary skill in the art, some improvements and modifications can be made without departing from the principles of the present disclosure, and these improvements and modifications are also regarded as a protection scope of the present disclosure.

1. A heating assembly, which is applied in an atomization device and configured for heating and atomizing liquid to be atomized, wherein the heating assembly comprises:
an oil guiding member defining a through hole cutting through the oil guiding member, and the through hole configured to guide atomized gas, generated on the oil guiding member, out of the oil guiding member; the oil guiding member comprising a connecting part, and the connecting part being located in the through hole, and the connecting part spanning the through hole, along a radial direction of the through hole; and
a heating member, embedded in one end of the oil guiding member; at least part of the heating member being disposed on the connecting part; the heating member being configured for heating the oil guiding member, so that the liquid to be atomized is atomized on an end face of the oil guiding member close to the heating member.

2. The heating assembly according to claim 1, wherein the oil guiding member comprises a main body part and an extension part that are coupled to each other; the through hole cuts through the extension part and the main body part in sequence; the main body part comprises a bearing surface and an atomizing surface arranged opposite to each other; the bearing surface comprises a connecting surface and a liquid absorbing surface that are coupled to each other; the extension part is fixedly arranged on the connecting surface; the connecting part is arranged at an opening of the through hole close to the atomizing surface; the liquid to be atomized flows to the atomizing surface through the liquid absorbing surface, and is atomized on the atomizing surface.

3. The heating assembly according to claim 2, wherein the heating member comprises a heating body and a positive electrode contact and a negative electrode contact electrically coupled to both ends of the heating body; the heating body is arranged on the atomizing surface in an arc shape or a curve shape; an arc corresponding to the heating body is greater than or equal to 180°, and at least part of the heating body is located on the connecting part.

4. The heating assembly according to claim 2, wherein the heating member comprises a heating body and a positive electrode contact and a negative electrode contact electrically coupled to both ends of the heating body; the positive electrode contact and the negative electrode contact are arranged on opposite sides of the through hole along the radial direction of the through hole; the positive electrode contact and the negative electrode contact are respectively located close to a first end and a second end of the connecting part; a first part of the heating body extends from the positive electrode contact, along the atomizing surface, to the second end of the connecting part; a second part of the heating body extends from the second end of the connecting part to the first end of the connecting part; a third part of the heating body extends from the first end of the connecting part, along the atomizing surface, to the second end of the connecting part and connects the negative electrode contact.

5. The heating assembly according to claim 2, wherein the extension part comprises a sleeve and at least one protrusion located on an outer peripheral surface of the sleeve; the at least one protrusion abuts against the atomizing surface; the sleeve, the at least one protrusion and the main body are integrally formed; the main body further defines a plurality of receiving grooves; openings of the receiving grooves are defined on the atomizing surface; at least part of bottom of the receiving grooves extends to the protrusion; the plurality of the receiving grooves are configured for receiving the positive electrode contact and the negative electrode contact respectively.

6. An atomization device, wherein, the atomization device comprises a heating assembly, the heating assembly comprises:
an oil guiding member defining a through hole cutting through the oil guiding member, and the through hole configured to guide atomized gas generated on the oil guiding member out of the oil guiding member; the oil guiding member comprising a connecting part, and the connecting part being located in the through hole, and the connecting part spanning the through hole, along a radial direction of the through hole; and
a heating member, embedded in one end of the oil guiding member; at least part of the heating member being disposed on the connecting part; the heating member being configured for heating the oil guiding member, so that the liquid to be atomized is atomized on an end face of the oil guiding member close to the heating member.

7. The atomization device according to claim 6, wherein the atomization device further comprises an atomizing rod and a sealing seat; the atomizing rod comprises a receiving seat; the heating assembly, the sealing seat and the receiving seat are sequentially sleeved from inside to outside; a part of the sealing seat is sealed between one side of the extension part away from the main body and the receiving seat, and another part of the sealing seat is sealed between a peripheral side wall of the extension part and the receiving seat, and another part of the sealing seat is sealed between a peripheral side wall of the main body and the receiving seat; a peripheral side wall of the extension part, the liquid absorbing surface, and an inner side wall of the sealing seat cooperatively form a receiving space; the receiving seat defines at least one liquid guiding hole; the sealing seat defines at least one liquid inlet hole; the at least one liquid guiding hole, the at least one liquid inlet hole and the receiving space are interconnected in sequence; the liquid to be atomized passes through the at least one liquid guiding hole, the at least one liquid inlet hole and the receiving space in sequence and then flows to the liquid absorbing surface.

8. The atomization device according to claim 7, wherein the atomizing rod further comprises a main rod body, which is integrally formed with the receiving seat; an inner cavity of the main rod body, a part of an inner cavity of the receiving seat, and a part of an inner cavity of the sealing seat and the through hole are sequentially interconnected; the atomized gas generated on the atomizing surface passes through the through hole, the part of the inner cavity of the sealing seat, the part of the inner cavity of the receiving seat, the part of the inner cavity of the receiving seat, and the inner cavity of the main rod body, and then flows out.

9. The atomization device according to claim 8, wherein the atomization device further comprises a suction nozzle and an oil cup; the suction nozzle is sleeved and sealed on a periphery of an end of the main rod body away from the receiving seat; the oil cup is sleeved on the periphery of the main rod body; one end of the oil cup is coupled to the suction nozzle hermetically, and the other end of the oil cup is coupled to a periphery of the receiving seat away from the main rod body; an inner side wall of the oil cup and an outer side wall of the atomizing rod cooperatively form a liquid storage cavity for storing the liquid to be atomized; the at least one liquid guiding hole is interconnected with the liquid storage cavity.

10. The atomization device according to claim 9, wherein the atomization device further comprises an atomization base, an atomization outer ring and an oil absorbing member; one end of the atomization base is received in an inner cavity of the receiving seat and abuts against the sealing seat, and the other end extends out of the receiving seat; an inner cavity of the atomization base is interconnected to the through hole; the oil absorbing member is arranged in the atomization base, and is configured to absorb un-atomized liquid that is to be atomized on the atomizing surface; the atomizing outer ring is fixedly arranged on a periphery of the end of the oil cup away from the suction nozzle; a periphery of an end of the receiving seat away from the main rod body, and a periphery of the atomization base.

11. The atomization device according to claim 10, wherein the atomization device further comprises a battery assembly, the battery assembly is electrically coupled to the heating member, the battery assembly is coupled to an end of the atomization base extending out of the atomization outer ring.

12. The heating assembly according to claim 3, wherein the extension part comprises a sleeve and at least one protrusion located on an outer peripheral surface of the sleeve; the at least one protrusion abuts against the atomizing surface; the sleeve, the at least one protrusion and the main body are integrally formed; the main body further defines a plurality of receiving grooves; openings of the receiving grooves are defined on the atomizing surface; at least part of bottom of the receiving grooves extends to the protrusion; the plurality of the receiving grooves are configured for receiving the positive electrode contact and the negative electrode contact respectively.

13. The atomization device according to claim 6, wherein the oil guiding member comprises a main body part and an extension part that are coupled to each other; the through hole cuts through the extension part and the main body part in sequence; the main body part comprises a bearing surface and an atomizing surface arranged opposite to each other; the bearing surface comprises a connecting surface and a liquid absorbing surface that are coupled to each other; the extension part is fixedly arranged on the connecting surface; the connecting part is arranged at an opening of the through hole close to the atomizing surface; the liquid to be atomized flows to the atomizing surface through the liquid absorbing surface, and is atomized on the atomizing surface.

14. The atomization device according to claim 13, wherein the heating member comprises a heating body and a positive electrode contact and a negative electrode contact electrically coupled to both ends of the heating body; the heating body is arranged on the atomizing surface in an arc shape or a curve shape; an arc corresponding to the heating body is greater than or equal to 180°, and at least part of the heating body is located on the connecting part.

15. The atomization device according to claim 13, wherein the heating member comprises a heating body and a positive electrode contact and a negative electrode contact electrically coupled to both ends of the heating body; the positive electrode contact and the negative electrode contact are arranged on opposite sides of the through hole along the radial direction of the through hole; the positive electrode contact and the negative electrode contact are respectively located close to a first end and a second end of the connecting part; a first part of the heating body extends from the positive electrode contact along the atomizing surface to the second end of the connecting part; a second part of the heating body extends from the second end of the connecting part to the first end of the connecting part; a third part of the heating body extends from the first end of the connecting part along the atomizing surface to the second end of the connecting part and connects the negative electrode contact.

16. The atomization device according to claim 13, wherein the extension part comprises a sleeve and at least one protrusion located on an outer peripheral surface of the sleeve; the at least one protrusion abuts against the atomizing surface; the sleeve, the at least one protrusion and the main body are integrally formed; the main body further defines a plurality of receiving grooves; openings of the receiving grooves are defined on the atomizing surface; at least part of bottom of the receiving grooves extends to the protrusion; the plurality of the receiving grooves are configured for receiving the positive electrode contact and the negative electrode contact respectively.

17. The atomization device according to claim 14, wherein the extension part comprises a sleeve and at least one protrusion located on an outer peripheral surface of the sleeve; the at least one protrusion abuts against the atomizing surface; the sleeve, the at least one protrusion and the main body are integrally formed; the main body further defines a plurality of receiving grooves; openings of the receiving grooves are defined on the atomizing surface; at least part of bottom of the receiving grooves extends to the protrusion; the plurality of the receiving grooves are configured for receiving the positive electrode contact and the negative electrode contact respectively.
